(19) United States
(12) Patent Application Publication
(10) Pub. No.: US 2009/0030005 A1
(43) Pub. Date: Jan. 29, 2009

(54) Combinations for the treatment of cancer

(75) Inventors: Alexander Kamb, Hillsborough, CA (US); Astrid Ruefli-Brasse, San Mateo, CA (US)

Correspondence Address: AMGEN INC., MAIL STOP 28-2-C, ONE AMGEN CENTER DRIVE, THOUSAND OAKS, CA, 91320-1799, US

(73) Assignee: AMGEN INC., Thousand Oaks, CA (US)

(21) Appl. No.: 12/218,865

(22) Filed: Jul. 17, 2008

Related U.S. Application Data
(60) Provisional application No. 60/961,278, filed on Jul. 19, 2007.

(51) Int. Cl. A61K 31/4985; A61K 31/453; C12Q 1/68; A61P 35/00; A61K 31/197

(52) U.S. Cl. 514/249; 514/320; 514/564; 435/6

(57) ABSTRACT

This invention is in the field of pharmaceutical agents and specifically relates to combinations, compositions, uses and methods for treating cancer.

This application claims the benefit of U.S. Provisional Application No. 60/961,278, filed Jul. 19, 2007, which is hereby incorporated herein by reference in its entirety.

### FIELD OF THE INVENTION

This invention is in the field of pharmaceutical agents and specifically relates to combinations, compositions, uses and methods for treating cancer.

### BACKGROUND OF THE INVENTION

Cyclin dependent kinases (cdks) play a key role in regulating the cell cycle. They consist of a catalytic subunit (the kinase) and a regulatory subunit (the cyclin). Kinase subunits (e.g. cdk 1-9) have been identified along with several regulatory subunits (cyclins A-H).

Each kinase associates with a specific regulatory partner and together make up the active catalytic moiety. Each transition of the cell cycle is regulated by a particular cdk complex: G11/S by cdk2/cyclin E, cdk4/cyclin D1 and cdk6/cyclin D2; S/G2 by cdk2/cyclin A and cdk1/cyclin A; G2 /M by cdk1/B. The coordinated activity of these kinases guides the individual cells through the replication process and ensures the vitality of each subsequent generation.

A link between tumor development and cdk related malfunctions has been identified. Over expression of the cyclin regulatory proteins and subsequent kinase hyperactivity have been linked to several types of cancers (Jiang, Proc. Natl. Acad. Sci. USA 90:9026-9030, 1993; Wang, Nature 343:555-557, 1990). Endogenous, highly specific protein inhibitors of cdks are frequently homozygously deleted in tumors and were found to have a major effect on cellular proliferation (Kamb et al, Science 264:436-440, 1994; Beach, Nature 336:701-704, 1993). These inhibitors include p¹⁶INK4 (an inhibitor of cdk4/D1), p²¹CIP1 (a general cdk inhibitor), and p²⁷KIP1 (a specific cdk2/E inhibitor). These proteins help to regulate the cell cycle through specific interactions with their corresponding cdk complexes. Cells deficient in these inhibitors are prone to unregulated growth and tumor formation.

Tumors with homozygous deletion of p¹⁶INK4 also have frequent deletions in a neighboring gene, methylthioadenosine phosphorylase (MTAP), due to their close proximity on chromosome 9. MTAP is a key enzyme in the salvage of adenine. The critical pool of adenosine is maintained by a complicated process, that conceptually involves two distinct pathways: de novo synthesis and salvage synthesis. Most ATP is created through aminination of inosine 5′-monophosphate (IMP) via the de dovo purine nucleotide cycle. De novo purine synthesis is a well-defined biochemical pathway. Adenylosuccinate lyase (AdSL) is an enzyme in this pathway that acts at two different steps. AdSL converts 5-aminoimidazole-4-(N-succinylocarbxamide) ribotide (SACAIR) to 5-aminoimidazole-4-carboxamide ribotide (ACAIR) and adenylosuccinate (SAMP) to adenosine monophosphate (AMP). Another enzyme in the de novo pathway, adenylosuccinate synthase (AdSS) catalyzes the first committed step in the conversion of IMP to AMP, converting IMP to SAMP. Salvage of ATP occurs through a series of biosynthetic steps culminating in production of AMP from 5-deoxy-5-methylthioadenosine (MTA) by action of the enzyme MTAP.

It is now found that some combinations of at least one agent that inhibits the de novo purine biosynthesis and at least one agent that inhibits CDK4 and/or CDK6 provides better results than one or the other inhibitor used alone.

### BRIEF DESCRIPTION OF THE DRAWINGS

FIG. 1 shows the dose response of a CDK inhibitor in MTAP+/+ or MTAP−/− MiaPaCa cells. [▪—MTAP^{-/-} cells; ▴—MTAP^{+/+} cells]

FIG. 2 shows the combination of a CDK inhibitor with alanosine in MTAP^{+/+} MiaPaCa cells with or without 20 μM adenine. [▪—CDK inhibitor alone; ▴—CDK inhibitor and alanosine; ▾—CDK inhibitor; alanosine and adenine; ♦—CDK inhibitor and adenine]

FIG. 3 shows the combination of a CDK inhibitor with alanosine in MTAP^{-/-} MiaPaCa cells with or without 20 μM adenine. [▪—CDK inhibitor alone; ▴—CDK inhibitor and alanosine; ▾—CDK inhibitor; alanosine and adenine; ♦—CDK inhibitor and adenine]

FIG. 4 shows the combination of a CDK inhibitor with alanosine in MTAP^{+/+} MiaPaCa cells with or without 20 μM MTA. [▪—CDK inhibitor alone; ▴—CDK inhibitor and alanosine; ▾—CDK inhibitor; alanosine and MTA; ♦—CDK inhibitor and MTA]

FIG. 5 shows the combination of a CDK inhibitor with alanosine in MTAP^{-/-} MiaPaCa cells with or without 20 μM MTA. [▪—CDK inhibitor alone; ▴—CDK inhibitor and alanosine; ▾—CDK inhibitor; alanosine and MTA; ♦—CDK inhibitor and MTA]

FIG. 6 shows the combination of a CDK inhibitor with thymidine (20 μM) with and without methotrexate (MTX, 20 nM (IC₂₀)) on MTAP^{+/+} MiaPaCa cells. [▪—CDK inhibitor; ▴—CDK inhibitor and MTX]

FIG. 7 shows the combination of a CDK inhibitor with thymidine (20 μM) with and without methotrexate (MTX, 20 nM (IC₂₀)) on MTAP^{-/-} MiaPaCa cells. [▪—CDK inhibitor; ▴—CDK inhibitor and MTX]

### DETAILED DESCRIPTION

The present invention is generally directed to compositions and methods for reducing tumor growth, and generally treating tumors in animals, including humans. The present invention is the determination that a combination of at least one agent that inhibits the de novo purine biosynthesis and at least one agent that inhibits CDK4 and/or CDK6 provides a beneficial effect. The results obtained indicate that targeting both CDK4/6 and de novo AMP biosynthesis has a heightened effect in tumors pre-selected for loss of p16 and MTAP. This result is unexpected because it has been assumed that CDK4/6 antagonism would be cytostatic, and might actually protect cells from agents that diminish AMP synthesis. Thus the present invention offers a surprising benefit from the combination of at least one agent that inhibits the de novo purine biosynthesis and at least one agent that inhibits CDK4 and/or CDK6, and that therapies which involve administration of combinations of these agents are beneficial in the treatment of cancer. The surprising benefit between the individual agents tested provide a number of unforeseen options for the treatment of tumors or cancers.

Inhibitors against enzymes in the de novo pathway will kill MTAP-deficient (MTAP^{-/-}) tumors, while leaving the salvage pathway intact in MTAP-positive (MTAP^{+/+}) cells, providing a source of ATP for normal tissues. The de novo purine biosynthesis pathway includes several key points for intervention. For example, adenylosuccinate synthetase (AdSS) and adenylosuccinate lyase (AdSL) catalyze the conversion of IMP to adenylsuccinate and AMP. AdSL also catalyses the conversion of succinylaminoimidazole-carboxide ribotide (SAICAR) to aminoimidazolecarboxamide ribotide (AICAR). Therefore an agent that inhibits AdSS is included in this invention. Alternatively, an agent that inhibits AdSL is included in this invention.

MTAP functions in both purine and polyamine metabolism in rapidly dividing cells. Tumors that have lost MTAP rely on the de novo pathway for ATP production. The absence of MTAP distinguishes some leukemic cells in vivo from their nonmalignant counterparts. Many other tumors lack MTAP due to homozygous deletion.

Inhibitors against enzymes in the de novo pathway include an adenine biosynthesis inhibitor such as alanosine, SDX-102 (the L-isomer of alanosine, which was under development by Cephalon for the treatment of cancer and the like). Formulations for L-alanosine are described in US2006/0041013. Methotrexate (MTX) has been found to interfere with de novo purine synthesis by inhibiting dhfr and reducing the available folate required for several of the enzymatic reactions involved in the biochemical pathway. Other inhibitors of de novo purine synthesis are described in U.S. Pat. No. 7,157,551.

One of the most important and fundamental processes in biology is the division of cells mediated by the cell cycle. This process ensures the controlled production of subsequent generations of cells with defined biological function. It is a highly regulated phenomenon and responds to a diverse set of cellular signals both within the cell and from external sources. A complex network of tumor promoting and suppressing gene products are key components of this cellular signaling process. Over expression of the tumor promoting components or the subsequent loss of the tumor suppressing products will lead to unregulated cellular proliferation and the generation of tumors.

Protein kinases, in particular, CDK, play a role in the regulation of cellular proliferation. Therefore, CDK inhibitors would be useful in the treatment of cell proliferative disorders such as cancer, familial adenomatosis polyposis, and vascular smooth cell proliferation. CDK4/6 inhibitors are especially attractive as anti-cancer therapies because of somatic mutations that are believed to activate (or more precisely, relieve their inhibition) which occur in a high proportion of cancers.

Agents known to inhibit CDK4 and or CDK6 include:
P-276-00 (a selective inhibitor of CDK4-cyclin D1, under development by Nicholas Piramal for the treatment of cancer);
GW-491619 (a CDK4 inhibitor, under development by GlaxoSmithKline for the treatment of cancer);
NU-6027 (a cyclin dependent kinase (CDK) inhibitor under investigation by AstraZeneca for use in cancer);
AG-12275 (a selective CDK4 inhibitor under investigation by Pfizer for the treatment of cancer);
AG-12286 (a broad-spectrum CDK4 inhibitor under investigation by Pfizer for the treatment of cancer);
PD-0166285 (a cyclin A-mediated inhibitor of CDK4 under investigation by Pfizer for the treatment of cancer);
PD-0332991 (a highly-specific CDK4/6 inhibitor, under development by Pfizer for the treatment of cancer);

Alvocidib (flavopiridol; HMR-1275, an inhibitor of Cdk4 under development by Sanofi-Aventis as an anticancer agent).

Other CDK4/6 inhibitors are described in WO 03/062236. Examples of such inhibitors include:
8-Cyclopentyl-2-(pyridin-2-ylamino)-8H-pyrido[2,3-d]pyrimidin-7-one,
6-Bromo-8-cyclopentyl-2-(5-piperazin-1-yl-pyridin-2-ylamino)-8H-pyrido[2,3-d]pyrimidin-7-one hydrochloride,
8-Cyclopentyl-6-ethyl-2-(5-piperazin-1-yl-pyridin-2-ylamino)-8H-pyrido[2,3-d]pyrimidin-7-one hydrochloride,
8-Cyclopentyl-7-oxo-2-(5-piperazin-1-yl-pyridin-2-ylamino)-7,8-dihydro-pyrido[2,3-d]pyrimidine-6-carboxylic acid ethyl ester hydrochloride,
6-Amino-8-cyclopentyl-2-(5-piperazin-1-yl-pyridin-2-ylamino)-8H-pyrido[2,3-d]pyrimidin-7-one hydrochloride,
6-Bromo-8-cyclopentyl-2-[5-((R)-1-methy-1-pyrrolidin-2-yl)-pyridin-2-ylamino]-8H-pyrido[2,3-d]pyrimidin-7-one hydrochloride,
6-Bromo-8-cyclohexyl-2-(pyridin-2-yl-amino)-8H-pyrido[2,3-d]pyrimidin-7-one,
6-Bromo-8-cyclopentyl-2-methyl-8H-pyrido[2,3-d]pyrimidin-7-one,
6-Bromo-8-cyclopentyl-5-methyl-2-(5-piperizin-1-yl-pyridin-2-ylamino)-8H-pyrido[2,3-d]pyrimidin-7-one,
8-Cyclopentyl-6-fluoro-2-(5-piperazin-1-yl-pyridin-2-ylamino)-8H-pyrido[2,3-d]pyrimidin-7-one hydrochloride,
8-Cyclopentyl-6-methyl-2-(5-piperazin-1-yl-pyridin-2-ylamino)-8H-pyrido[2,3-d]pyrimidin-7-one hydrochloride,
8-Cyclopentyl-6-isobutoxy-2-(5-piperazin-1-yl-pyridin-2-ylamino)-8H-pyrido[2,3-d]pyrimidin-7-one hydrochloride,
6-Benzyl-8-cyclopentyl-2-(5-piperazin-1-yl-pyridin-2-ylamino)-8H-pyrido[2,3-d]pyrimidin-7-one hydrochloride,
8-Cyclopentyl-6-hydroxymethyl-2-(5-piperazin-1-yl-pyridin-2-ylamino)-8H-pyrido[2,3-d]pyrimidin-7-one hydrochloride,
2-[5-(4-tert-Butoxycarbonyl-piperazin-1-yl)-pyridin-2-ylamino]-8-cyclopentyl-5-methyl-7-oxo-7,8-dihydro-pyrido[2,3-d]pyrimidine-6-carboxylic acid ethyl ester,
6-Acetyl-8-cyclopentyl-2-(5-piperazin-1-yl-pyridin-2-ylamino)-8H-pyrido[2,3-d]pyrimidin-7-one,
6-Acetyl-8-cyclopentyl-5-methyl-2-(5-piperazin-1-yl-pyridin-2-ylamino)-8H-pyrido[2,3-d]pyrimidin-7-one,
6-Bromo-8-cyclopentyl-5-methyl-2-(pyridin-2-ylamino)-8H-pyrido[2,3-d]pyrimidin-7-one,
6-Bromo-8-cyclopentyl-2-(pyridin-2-ylamino)-8H-pyrido[2,3-d]pyrimidin-7-one,
4-Cyclopentylamino-2-(5-piperazin-1-yl-pyridin-2-ylamino)-pyrimidine-5-carbonitrile,
N4-Cyclopentyl-5-nitro-N-2-(5-piperazin-1-yl-pyridin-2-yl)-pyrimidine-2,4-diamine,
4-Cyclopentylamino-2-(5-piperazin-1-yl-pyridin-2-ylamino)-pyrimidine-5-carbaldehyde,
4-Cyclopentylamino-2-(5-piperazin-1-yl-pyridin-2-ylamino)-pyrimidine-5-carboxylic acid ethyl ester,
4-Cyclopentylamino-2-(5-piperazin-1-yl-pyridin-2-ylamino)-pyrimidine-5-carboxylic acid methyl ester,
[4-Cyclopentylamino-2-(5-piperazin-1-yl-pyridin-2-ylamino)-pyrimidin-5-yl]-methanol,
1-[4-Cyclopentylamino-2-(5-piperazin-1-yl-pyridin-2-ylamino)-pyrimidin-5-yl]-ethanone,
3-[4-Cyclopentylamino-2-(5-piperazin-1-yl-pyridin-2-ylamino)-pyrimidin-5-yl]-but-2-enoic acid ethyl ester,
4-Amino-2-(5-piperazin-1-yl-pyridin-2-ylamino)-pyrimidine-5-carbonitrile,
5-Nitro-N-2-(5-piperazin-1-yl-pyridin-2-yl)-pyrimidine-2,4-diamine,
4-Amino-2-(5-piperazin-1-yl-pyridin-2-ylamino)-pyrimidine-5-carbaldehyde,
4-Amino-2-(5-piperazin-1-yl-pyridin-2-ylamino)-pyrimidine-5-carboxylic acid ethyl ester,
4-Amino-2-(5-piperazin-1-yl-pyridin-2-ylamino)-pyrimidine-5-carboxylic acid methyl ester,
[4-Amino-2-(5-piperazin-1-yl-pyridin-2-ylamino)-pyrimidin-5-yl]-methanol,
1-[4-Amino-2-(5-piperazin-1-yl-pyridin-2-ylamino)-pyrimidin-5-yl]-ethanone,
3-[4-Amino-2-(5-piperazin-1-yl-pyridin-2-ylamino)-pyrimidin-5-yl]-but-2-enoic acid ethyl ester,
4-Cyclopentylamino-2-(5-pyrrolidin-1-yl-pyridin-2-ylamino)-pyrimidine-5-carbonitrile,
N2-[5-(3-Amino-pyrrolidin-1-yl)-pyridin-2-yl]-N-4-cyclopentyl-5-nitro-pyrimidine-2,4-diamine,
4-Cyclopentylamino-2-(5-morpholin-4-yl-pyridin-2-ylamino)-pyrimidine-5-carbaldehyde,
4-Cyclopentylamino-2-(3,4,5,6-tetrahydro-2H-[1,3′]bipyridinyl-6′-ylamino)-pyrimidine-5-carboxylic acid ethyl ester,
4-Cyclopentylamino-6-methyl-2-(5-piperazin-1-yl-pyridin-2-ylamino)-pyrimidine-5-carboxylic acid methyl ester,
{2-[5-(Bis-methoxymethyl-amino)-pyridin-2-ylamino]-4-cyclopentylamino-pyrimidin-5-yl}-methanol,
1-[4-Benzylamino-2-(5-piperazin-1-yl-pyridin-2-ylamino)-pyrimidin-5-yl]-ethanone,
4-[4-Cyclopentylamino-2-(5-piperazin-1-yl-pyridin-2-ylamino)-pyrimidin-5-yl]-pent-3-en-2-one,
4-Amino-2-(pyridin-2-ylamino)-pyrimidine-5-carbonitrile, 5-Nitro-N-2-pyridin-2-yl-pyrimidine-2,4-diamine,
4-Amino-2-(pyridin-2-ylamino)-pyrimidine-5-carbaldehyde,
4-Amino-2-(pyridin-2-ylamino)-pyrimidine-5-carboxylic acid ethyl ester,
5-Bromo-N-2-(5-piperazin-1-yl-pyridin-2-yl)-pyrimidine-2,4-diamine,
[4-Amino-2-(5-morpholin-4-yl-pyridin-2-ylamino)-pyrimidin-5-yl]-methanol,
1-[4-Amino-2-(5-morpholin-4-yl-pyridin-2-ylamino)-pyrimidin-5-yl]-ethanone,
[6-(5-Acetyl-4-amino-pyrimidin-2-ylamino)-pyridin-3-yloxy]-acetic acid,
4-Cyclopentylamino-2-(4-hydroxymethyl-5-pyrrolidin-1-yl-pyridin-2-ylamino)-pyrimidine-5-carbonitrile,
N2-[5-(3-Amino-pyrrolidin-1-yl)-6-chloro-pyridin-2-yl]-N-4-cyclopentyl-5-nitro-pyrimidine-2,4-diamine,
2-(5-Bromo-pyridin-2-ylamino)-4-cyclopentylamino-pyrimidine-5-carbaldehyde,
4-Cyclopentylamino-2-(1H-pyrrolo[3,2-b]pyridin-5-ylamino)-pyrimidine-5-carboxylic acid ethyl ester,
4-Cyclopentylamino-2-(4,6-dichloro-5-piperazin-1-yl-pyridin-2-ylamino)-6-methyl-pyrimidine-5-carboxylic acid methyl ester,
2-(2-{5-[Bis-(2-methoxy-ethyl)-amino]-pyridin-2-ylamino}-4-cyclopentylamino-pyrimidin-5-yl)-2-methyl-propan-1-ol,
1-[4-Phenylamino-2-(5-piperazin-1-yl-pyridin-2-ylamino)-pyrimidin-5-yl]-ethanone,
4-[4-(3-Hydroxy-cyclopentylamino)-2-(5-piperazin-1-yl-pyridin-2-ylamino)-pyrimidin-5-yl]-pent-3-en-2-one,
4-[5-Cyano-2-(pyridin-2-ylamino)-pyrimidin-4-ylamino]-cyclohexanecarboxylic acid,
2-(4-Amino-5-nitro-pyrimidin-2-ylamino)-isonicotinic acid,
4-Amino-6-methyl-2-(pyridin-2-ylamino)-pyrimidine-5-carbaldehyde,
5-Iodo-N-2-pyridin-2-yl-pyrimidine-2,4-diamine,
N-[5-Bromo-2-(5-piperazin-1-yl-pyridin-2-ylamino)-pyrimidin-4-yl]-acrylamide,
N2-(5-piperazin-1-yl-pyridin-2-yl)-5-prop-1-ynyl-pyrimidine-2,4-diamine,
5-[2-(4-Fluoro-phenyl)-ethyl]-N-2-(5-piperazin-1-yl-pyridin-2-yl)-pyrimidine-2,4-diamine,
[6-(4-Amino-5-propenyl-pyrimidin-2-ylamino)-pyridin-3-yloxy]-acetic acid,
5-Bromo-N-4-cyclopentyl-N-2-(5-pyrrolidin-1-yl-pyridin-2-yl)-pyrimidine-2,4-diamine,
N2-[5-(3-Amino-pyrrolidin-1-yl)-6-chloro-pyridin-2-yl]-5-bromo-N-4-cyclopentyl-pyrimidine-2,4-diamine,
5-Bromo-N4-cyclopentyl-N-2-(5-piperazin-1-yl-pyridin-2-yl)-pyrimidine-2,4-diamine,
5-Bromo-N-4-cyclopentyl-N-2-(1H-pyrrolo[3,2-b]pyridin-5-yl)-pyrimidine-2,4-diamine,
5-Bromo-N4-cyclopentyl-N-2-(4,6-dichloro-5-piperazin-1-yl-pyridin-2-yl)-6-methyl-pyrimidine-2,4-diamine,
N2-{5-[Bis-(2-methoxy-ethyl)-amino]-pyridin-2-yl}-5-bromo-N-4-cyclopentyl-pyrimidine-2,4-diamine,
5-Bromo-N-4-phenyl-N-2-(5-piperazin-1-yl-pyridin-2-yl)-pyrimidine-2,4-diamine,
3-[5-Bromo-2-(5-piperazin-1-yl-pyridin-2-ylamino)-pyrimidin-4-ylamino]-cyclopentanol,
N4-Cyclopentyl-5-iodo-N-2-(5-pyrrolidin-1-yl-pyridin-2-yl)-pyrimidine-2,4-diamine,
N2-[5-(3-Amino-pyrrolidin-1-yl)-6-chloro-pyridin-2-yl]-N-4-cyclopentyl-5-iodo-pyrimidine-2,4-diamine,
N4-Cyclopentyl-5-iodo-N-2-(5-piperazin-1-yl-pyridin-2-yl)-pyrimidine-2,4-diamine,
N4-Cyclopentyl-5-iodo-N-2-(1H-pyrrolo[3,2-b]pyridin-5-yl)-pyrimidine-2,4-diamine,
4-[6-(5-Bromo-4-cyclopentylamino-pyrimidin-2-ylamino)-pyridin-3-yl]-piperazine-1-carboxylic acid tert-butyl ester,
4-[6-(4-Cyclopentylamino-5-formyl-pyrimidin-2-ylamino)-pyridin-3-yl]-piperazine-1-carboxylic acid tert-butyl ester,
4-[6-(5-Acetyl-4-cyclopentylamino-pyrimidin-2-ylamino)-pyridin-3-yl]-piperazine-1-carboxylic acid tert-butyl ester,
2-[5-(4-tert-Butoxycarbonyl-piperazin-1-yl)-pyridin-2-ylamino]-4-cyclopentylamino-pyrimidine-5-carboxylic acid ethyl ester,
N-Cyclopentyl-N′-(5-piperazin-1-yl-pyridin-2-yl)-pyrimidine-4,6-diamine,
N-Isopropyl-N′-(5-piperazin-1-yl-pyridin-2-yl)-pyrimidine-4,6-diamine,
4-[6-(6-Cyclopentylamino-pyrimidin-4-ylamino)-pyridin-3-yl]-piperazine-1-carboxylic acid tert-butyl ester,
N-[5-(3-Amino-pyrrolidin-1-yl)-pyridin-2-yl]-N′-cyclopentyl-pyrimidine-4,6-diamine,
4-{6-[4-Cyclopentylamino-5-(1-methyl-3-oxo-but-1-enyl)-pyrimidin-2-ylamino]-pyridin-3-yl}-piperazine-1-carboxylic acid tert-butyl ester,
N-Cyclopentyl-N′-(5-piperazin-1-yl-pyridin-2-yl)-[1,3,5]triazine-2,4-diamine,
1-[4-Cyclopentylamino-2-(5-piperazin-1-yl-pyridin-2-ylamino)-pyrimidin-5-yl]-ethanone,
5-Bromo-N4-cyclopentyl-N-2-(5-piperazin-1-yl-pyridin-2-yl)-pyridine-2,4-diamine,
4-Cyclopentylamino-6-(5-piperazin-1-yl-pyridin-2-ylamino)-nicotinonitrile,
N4-Cyclopentyl-5-nitro-N-2-(5-piperazin-1-yl-pyridin-2-yl)-pyridine-2,4-diamine,
4-Cyclopentylamino-6-(5-piperazin-1-yl-pyridin-2-ylamino)-pyridine-3-carbaldehyde,
4-Cyclopentylamino-6-(5-piperazin-1-yl-pyridin-2-ylamino)-nicotinic acid ethyl ester,
4-Cyclopentylamino-6-(5-piperazin-1-yl-pyridin-2-ylamino)-nicotinic acid methyl ester,
[4-Cyclopentylamino-6-(5-piperazin-1-yl-pyridin-2-ylamino)-pyridin-3-yl]-methanol,
1-[4-Cyclopentylamino-6-(5-piperazin-1-yl-pyridin-2-ylamino)-pyridin-3-yl]-ethanone,
3-[4-Cyclopentylamino-6-(5-piperazin-1-yl-pyridin-2-ylamino)-pyridin-3-yl]-but-2-enoic acid ethyl ester,
(5-Cyclopentyl-5,6-dihydro-pyrido[2,3-e][1,2,4]triazin-3-yl)-(5-piperazin-1-yl-pyridin-2-yl)-amine,
(8-Cyclopentyl-7-methoxy-quinazolin-2-yl)-(5-piperazin-1-yl-pyridin-2-yl)-amine,
(8-Cyclopentyl-7-methoxy-pyrido[3,2-d]pyrimidin-2-yl)-(5-piperazin-1-yl-pyridin-2-yl)-amine,
6-Acetyl-8-cyclopentyl-2-(5-piperazin-1-yl-pyridin-2-ylamino)-8H-pyridin-7-one,
3-Acetyl-1-cyclopentyl-7-(5-piperazin-1-yl-pyridin-2-ylamino)-1H-pyrido[3,4-b]pyrazin-2-one,
1-Cyclopentyl-3-ethyl-4-methyl-7-(5-piperazin-1-yl-pyridin-2-ylamino)-3,4-dihydro-1H-pyrimido[4,5-d]pyrimidin-2-one,
1-Cyclopentyl-3-ethyl-4-methyl-7-(5-piperazin-1-yl-pyridin-2-ylamino)-3,4-dihydro-1H-pyrido[4,3-d]pyrimidin-2-one,
3-Acetyl-1-cyclopentyl-4-methyl-7-(5-piperazin-1-yl-pyridin-2-ylamino)-1H-[1,6]naphthyridin-2-one,
(9-Isopropyl-6-methyl-9H-purin-2-yl)-(5-piperazin-1-yl-pyridin-2-yl) -amine,
2-[9-Isopropyl-6-(5-piperazin-1-yl-pyridin-2-ylamino)-9H-purin-2-ylamino]-ethanol,
N2-(4-Amino-cyclohexyl)-9-cyclopentyl-N-6-(5-piperazin-1-yl-pyridin-2-yl)-9H-purine-2,6-diamine,
2-[9-Isopropyl-6-(5-piperazin-1-yl-pyridin-2-ylamino)-9H-purin-2-ylamino]-3-methyl-butan-1-ol,
(1-Isopropyl-4-methyl-1H-pyrazolo[3,4-d]pyrimidin-6-yl)-(5-piperazin-1-yl-pyridin-2-yl)-amine,
2-[1-Isopropyl-4-(5-piperazin-1-yl-pyridin-2-ylamino)-1H-pyrazolo[3,4-d]pyrimidin-6-ylamino]-ethanol,
N6-(4-Amino-cyclohexyl)-1-cyclopentyl-N4-(5-piperazin-1-yl-pyridin-2-yl)-1H-pyrazolo[3,4-d]pyrimidine-4,6-diamine,
2-[1-Isopropyl-4-(5-piperazin-1-yl-pyridin-2-ylamino)-1H-pyrazolo[3,4-d]pyrimidin-6-ylamino]-3-methyl-butan-1-ol,
5-Cyclopentyl-7-(1-hydroxy-ethyl)-8-methyl-3-(5-piperazin-1-yl-pyridin-2-ylamino)--5H-pyrido[3,2-c]pyridazin-6-one,
5-Cyclopentyl-8-methyl-3-(5-piperazin-1-yl-pyridin-2-ylamino)-5H-pyrido[3,2-c]pyridazin-6-one,
7-Benzyl-5-cyclopentyl-3-(5-piperazin-1-yl-pyridin-2-ylamino)-5H-pyrido[3,2-c]pyridazin-6-one,
[5-(1,1-Dioxo-116-thiomorpholin-4-yl)-pyridin-2-yl]-(4-isopropyl-3-methoxy-2-methyl-[1,7]naphthyridin-6-yl)-amine,
(2-Ethyl-4-isopropyl-3-methoxy-[1,7]naphthyridin-6-yl)-pyridin-2-yl-amine,
(2,4-Diisopropyl-3-methoxy-[1,7]naphthyridin-6-yl)-(5-isopropenyl-pyridin-2-yl)-amine,
[4-(2-Ethylamino-pyridin-4-yl)-pyrimidin-2-yl]-(5-piperazin-1-yl-pyridin-2-yl)-amine,
[4-(5-Ethyl-2-methylamino-pyridin-4-yl)-pyrimidin-2-yl]-(5-morpholin-4-yl-pyridin-2-yl)-amine,
[5-Methoxy-4-(2-methylamino-pyridin-4-yl)-pyrimidin-2-yl]-(5-morpholin-4-yl-pyridin-2-yl)-amine, and 5-Fluoro-N-4-isopropyl-N-2-(5-piperazin-1-yl-pyridin-2-yl)-pyrimidine-2,4-diamine.

CDK4 inhibitors can be prepared based on the descriptions found in U.S. Pat. No. 6,689,864, PCT Patent Publication No. WO08/007123, PCT Patent Publication No. WO07/140222, PCT Patent Publication No. WO06/106046, PCT Patent Publication No. WO03/062236, PCT Patent Publication No. WO05/005426, PCT Patent Publication No. WO99/21845; PCT Patent Publication No. WO06/097449, PCT Patent Publication No. WO06/097460, PCT Patent Publication No. WO99/02162, and PCT Patent Publication No. WO99/50251. For a discussion of standard CDK4 assays, see D. W. Fry et al., J. Biol. Chem. (2001) 16617-16623. Assays for CDK6 inhibitors is similar to that described substituting expressed CDK6 protein.

Other specific CDK inhibitors are described in EP1250353, WO02/96888, WO03/076437, WO03/76436, WO03/76434, and WO01/64368.

It has been found, because the genes for p16 and MTAP are closely linked on the chromosome, the large majority of tumors that have undergone activation of CDK4/6 through homozygous deletion of the cognate CDK inhibitor, p16, have also lost the MTAP gene. This creates a dependency in the tumors on de novo synthesis of AMP. Therefore, a simple diagnostic test can target tumors that are (i) dependent on CDK4/6; and (ii) dependent on de novo AMP synthesis. Thus, another aspect of the present invention comprises treatment of a cancer that activates a CDK protein such as CDK4/6, that also under-expresses MTAP.

The invention also comprises usage of a rescue substrate, such as MTA, 9-β-D-erythrofuranosyladenine (EFA), adenine, 5′-deoxyadenosine, or the like. U.S. Pat. Nos. 5,840,505 and 6,214,571 describe the treatment of alanosine treated cells with MTA. WO03/074083 describes various MTA derivatives that should be useful as rescue substrates.

The invention also relates to treatment of neoplasia including cancer and metastasis, including, but not limited to: carcinoma such as cancer of the bladder, breast, colon (including colorectal cancer), kidney, head and neck, liver, lung (including non-small cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g. soft tissue and bone); tumors of the central and peripheral nervous system (including astrocytoma, neuroblastoma, glioma and schwannomas); and other tumors (including melanoma, seminoma, teratocarcinoma, osteosarcoma, xenoderoma pigmentosum, keratoctanthoma, thyroid follicular cancer and Kaposi's sarcoma).

The invention also relates to treatment of neoplasias that are MTAP deficient. Possible patients can be tested to determine whether they have cancer cells that are homozygous for MTAP deficiency. MTAP deficiency also includes cells where the MTAP expression and or activity is partially reduced, substantially reduced or eliminated. Such deficiency means that the cells ability to replenish the adenine pool is negatively impacted.

The present invention includes a method for prognostic or diagnostic assessment of a neoplastic disorder in a subject, comprising: a) preparing a sample of nucleic acids from a specimen obtained from the subject; b) contacting the sample with a panel of nucleic acid segments consisting of at least 2 members from the group consisting of p16, CDK4, CDK6, and MTAP to detect the levels of the panel segments; c) evaluating the sample against a reference standard to determine the magnitude of change in the amounts of the at least 2 members present in the sample; and d) correlating the magnitude of change with the presence or resolution of the disorder.

The invention also relates to a method for prognostic or diagnostic assessment wherein the detection identifies a disorder that is likely to respond to a composition comprising at least one de novo purine biosynthesis inhibitor and at least one CDK inhibitor.

The invention also relates to the use of the combination of at least one de novo purine biosynthesis with at least one CDK4 and/or CDK6 inhibitor in adjuvant or neoadjuvant chemotherapy, with or without radiation, for the treatment of neoplasia. “Adjuvant chemotherapy” is defined as the continued treatment after either intensive cycles of chemotherapy and/or radiation, or alternatively after surgery to remove tumors. Alternatively the term describes the use of drugs as additional treatment for patients with cancers that are thought to have spread outside their original sites. Neo-adjuvant therapy is defined as intensive cycles of chemotherapy and/or radiation given to reduce the size of tumor before a definitive surgery. Such adjuvant or neo-adjuvant chemotherapy +/− radiation relates to the treatment of neoplasia including, but not limited to: carcinoma of the breast, colon, lung, and head and neck.

The invention is also directed to a method of administration of the combination. More particularly the active agents of the combination therapy are administered sequentially in either order or simultaneously. When the active agents are administered simultaneously, one skilled in the art will understand that the second agent can be administered some time after the first agent. The particular period of delay is dependent on the particular pharmacokinetic and formulation parameters of the active agent. The invention also relates to treatment wherein the de novo purine synthesis inhibitor is pre-dosed (administered first), followed by treatment with the CDK4 or CDK6 inhibitor. Alternatively the pre-dose may occur 24-48 hours prior to the treatment with the CDK4 or CDK6 inhibitor.

The invention also relates to a kit, wherein the inhibitors are disposed in separate containers.

The invention also relates to a kit according to any of the foregoing, further comprising integrally thereto or as one or more separate documents, information pertaining to the contents or the kit and the use of the inhibitors.

As used in relation to the invention, the term “treating” or “treatment” and the like should be taken broadly. They should not be taken to imply that an animal is treated to total recovery. Accordingly, these terms include amelioration of the symptoms or severity of a particular condition or preventing or otherwise reducing the risk of further development of a particular condition.

The term “comprising” is meant to be open ended, including the indicated component but not excluding other elements.

The phrase “therapeutically-effective” is intended to qualify the amount of each agent, which will achieve the goal of improvement in disorder severity and the frequency of incidence over treatment of each agent by itself, while avoiding adverse side effects typically associated with alternative therapies. For example, effective neoplastic therapeutic agents prolong the survivability of the patient, inhibit the rapidly-proliferating cell growth associated with the neoplasm, or effect a regression of the neoplasm.

It should be appreciated that methods of the invention may be applicable to various species of subjects, preferably mammals, more preferably humans.

As used herein, the compounds of the present invention include the pharmaceutically acceptable derivatives thereof.

Where the plural form is used for compounds, salts, and the like, this is taken to mean also a single compound, salt and the like.

The term “CDK inhibitor” means a compound that inhibits CDK4, CDK6 or both CDK4/CDK6.

The terms “cancer” and “cancerous” when used herein refer to or describe the physiological condition in mammals that is typically characterized by unregulated cell growth. Examples of cancer include but are not limited to, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer.

A “pharmaceutically-acceptable derivative” denotes any salt, ester of a compound of this invention, or any other compound which upon administration to a patient is capable of providing (directly or indirectly) a compound of this invention, or a metabolite or residue thereof.

The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, aistidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. When a basic group and an acid group are present in the same molecule, a compound of the invention may also form internal salts.

Currently, standard treatment of primary tumors consists of surgical excision followed by either radiation or IV administered chemotherapy. The typical chemotherapy regime consists of either DNA alkylating agents, DNA intercalating agents, CDK2 inhibitors, or microtubule poisons. The chemotherapy doses used are just below the maximal tolerated dose and therefore dose limiting toxicities typically include, nausea, vomiting, diarrhea, hair loss, neutropenia and the like.

There are large numbers of antineoplastic agents available in commercial use, in clinical evaluation and in pre-clinical development, which would be selected for treatment of neoplasia by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents.

A first family of antineoplastic agents which may be used in combination with compounds of the present invention consists of antimetabolite-type/thymidilate synthase inhibitor antineoplastic agents. Suitable antimetabolite antineoplastic agents may be selected from but not limited to the group consisting of 5-FU, fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

A second family of antineoplastic agents which may be used in combination with compounds of the present invention consists of alkylating-type antineoplastic agents. Suitable alkylating-type antineoplastic agents may be selected from but not limited to the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which may be used in combination with compounds of the present invention consists of antibiotic-type antineoplastic agents. Suitable antibiotic-type antineoplastic agents may be selected from but not limited to the group consisting of Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with compounds of the present invention consists of a miscellaneous family of antineoplastic agents, including tubulin interacting agents, topoisomerase II inhibitors, topoisomerase I inhibitors and hormonal agents, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534.

The combination of the present invention comprises a composition of the present invention in combination with at least one anti-tumor agent. Agents are inclusive of, but not limited to, in vitro synthetically prepared chemical compositions, antibodies, antigen binding regions, radionuclides, and combinations and conjugates thereof. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-tumor agents include HERCEPTIN™ (trastuzumab), which may be used to treat breast cancer and other forms of cancer, and RITUXAN™ (rituximab), ZEVALIN™ (ibritumomab tiuxetan), and LYMPHOCIDE™ (epratuzumab), which may be used to treat non-Hodgkin's lymphoma and other forms of cancer, GLEEVAC™ which may be used to treat chronic myeloid leukemia and gastrointestinal stromal tumors, and BEXXAR™ (iodine 131 tositumomab) which may be used for treatment of non-Hodgkins's lymphoma.

Exemplary anti-angiogenic agents include ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

Other anti-angiogenic agents include Campath, IL-8, B-FGF, Tek antagonists (Ceretti et al., US Publication No. 2003/0162712; U.S. Pat. No. 6,413,932), anti-TWEAK agents (e.g., specifically binding antibodies or antigen binding regions, or soluble TWEAK receptor antagonists; see, Wiley, U.S. Pat. No. 6,727,225), ADAM distintegrin domain to antagonize the binding of integrin to its ligands (Fanslow et al., US Publication No. 2002/0042368), specifically binding anti-eph receptor and/or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide.(Merck KGaA, Germany, EPO 770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055,(Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 970070); ARGENT technology, (Ariad, USA); YIGSR-Stealth, (Johnson & Johnson, USA); fibrinogen-E fragment, (BioActa, UK); angiogenesis inhibitor, (Trigen, UK); TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (IVAX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); cancer therapy, (University of South Carolina, USA); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchernia, Australia); VEGF antagonist, (Regeneron, USA); rBPI 21 and BPI-derived antiangiogenic, (XOMA, USA); PI 88, (Progen, Australia); cilengitide (pNN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); oguanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aetema, Canada); vaccine, angiogenesis, (EntreMed, USA); urokinase plasminogen activator inhibitor, (Dendreon, USA); oglufanide (pINN), (Melmotte, USA); HIF-1 alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); MAb, KDR, (ImClone Systems, USA); MAb, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sima, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA);vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

Alternatively, the present combinations may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Alternatively, the present combinations may also be used with radiation. Alternatively, the present compounds may also be used in conjunction with agents used for hormonal therapy, such as for treatment of breast and prostate cancer. Examples include aromatase inhibitors (e.g. Arimidex (chemical name: anastrozole), Aromasin (chemical name: exemestane), and Femara (chemical name: letrozole)); Serms (selective estrogen-receptor modulators) such as tamoxifen; and ERDs (estrogen-receptor downregulators), e.g. Faslodex (chemical name: fulvestrant).

As will be appreciated, the dose of a combination of the present invention to be administered, the period of administration, and the general administration regime may differ between subjects depending on such variables as the severity of symptoms, the type of tumor to be treated, the mode of administration chosen, type of composition, size of a unit dosage, kind of excipients, the age and/or general health of a subject, and other factors well known to those of ordinary skill in the art.

Administration may include a single daily dose or administration of a number of discrete divided doses as may be appropriate. An administration regime may also include administration of one or more of the active agents, or compositions comprising same, as described herein. The period of administration may be variable.

It may occur for as long a period is desired.

Administration may include simultaneous administration of suitable agents or compositions or sequential administration of agents or compositions.

### FORMULATIONS

Also embraced within this invention is a class of pharmaceutical compositions comprising the active inhibitors in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, preferably from about 1 to 500 mg. A suitable daily dose for a human or other mammal may vary widely depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, preferably between about 0.01 and about 50 mg/kg, and more preferably about 0.01 and about 30 mg/kg body weight may be appropriate. The daily dose can be administered in one to four doses per day.

For therapeutic purposes, the active compounds of this invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (ie. Captisol), cosolvent solubilization (ie. propylene glycol) or micellar solubilization (ie. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The invention also provides kits comprising one or more de novo purine biosynthesis inhibitor with one or more CDK4 and/or CDK6 inhibitor in accordance with the foregoing. The inhibitors may be disposed in the kits in one or more containers. Each such container may contain separately or in admixture one or more de novo purine biosynthesis inhibitor and one or more CDK4 and/or CDK6 inhibitor in accordance with any of the foregoing. Typically, such kits are designed for medical use, and the inhibitors are comprised in pharmaceutically acceptable formulations. Also included are kits wherein the inhibitors are disposed in separate containers.

The kits are those that comprise integrally thereto or as one or more separate documents, information pertaining to the contents or the kit and the use of the inhibitors. Also among the kits are those wherein the compositions, if injectable, are formulated for reconstitution in a diluent. In this regard, kits further comprising one or more containers of sterile diluent are also included.

The present invention also includes kits wherein at least one of the inhibitors can be disposed in vials under partial vacuum sealed by a septum and suitable for reconstitution to form a formulation effective for parental administration. The present invention also includes kits wherein at least one of the inhibitors is in tablet form.

The present invention also include kits that provide single-dose packaging of one or more of the inhibitors.

The invention will now be further described with reference to the following non-limiting examples.

### EXAMPLES

### Example 1

Generation of MTAP^{+/+} and MTAP^{-/-} MiaPaCa-2 cells. MiaPaCa-2 pancreatic cells, which harbor a homozygous deletion in MTAP (MTAP^{-/-}) were obtained from ATCC. Cells were infected with either a control virus (pLPC) or a virus expressing MTAP (pLPC-MTAP). Infected cells were selected by puromycin resistance and maintained in 0.5 μg/ml puromycin. MTAP expression in MTAP^{+/+} MiaPaCa-2 cells was confirmed by QPCR.

### Example 2

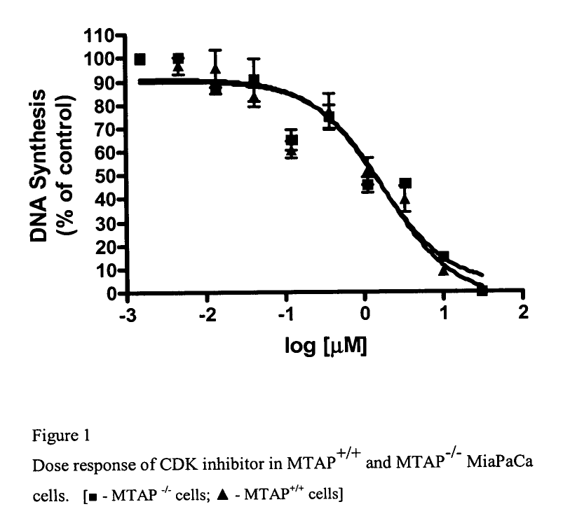

CDK4 inhibitor in MTAP^{+/+} and MTAP^{-/-} MiaPaCa-2 cells. The IC₅₀ of a CDK4 inhibitor was determined in the MTAP+/+ and MTAP−/− MiaPaCa-2 cells using a thymidine incorporation assay. MTAP^{-/-} and MTAP^{+/+} MiaPaCa-2 cells from Example 1 were seeded at 2×10⁴ cells/ml in 100 μl per well in Costar T plates (Amersham Biosciences). Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) was added to triplicate wells. ¹⁴C-Thymidine (0.1 μCi, Amersham) was added to each well for a final total volume of 200 μL. Plates were read on a beta counter for incorporation of the radioactive thymidine as a measure of cell viability at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor (calculated with GraphPrism 4) was 1.563 μM in the MTAP^{-/-} and 1.822 μM in the MTAP^{+/+} cells. See FIG. 1. This indicates that the IC₅₀'s for the compound is relatively consistent in both cell lines.

Similarly the IC₂₀ of alanosine (1.7 μM) and of MTX (2 nM) were determined in the same assay. Alanosine or MTX were then dosed at the IC₂₀ concentrations together with a dose response of the CDK4 inhibitor with or without adenine or MTA.

### Example 3

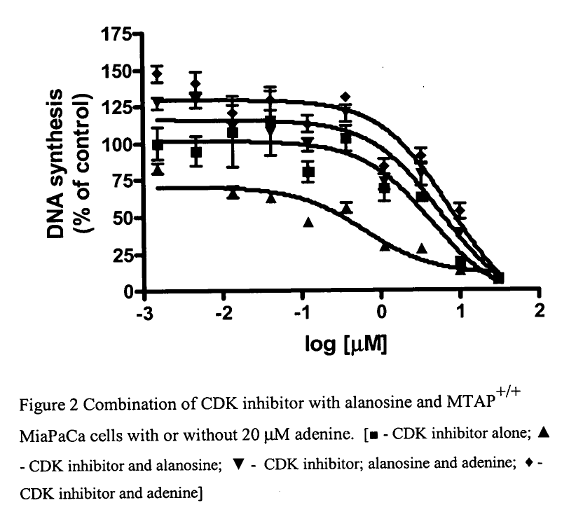

Combination of de novo purine biosynthesis inhibitor and CDK4 inhibitor in MTAP^{+/+} cells with adenine. MTAP^{+/+} MiaPaCa-2 cells from Example 1 were seeded at 2×10⁴ cells/ml in 100 μl per well in Costar T plates (Amersham Biosciences). Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) was added to triplicate wells with or without 1.7 μM (IC₂₀) of alanosine. Some cells were also treated with 20 μM adenine (Sigma) as rescue substrate. ¹⁴C-Thymidine (0.1 μCi, Amersham) was added to each well for a final total volume of 200 μL. Plates were read for incorporation of the radioactive thymidine as a measure of cell viability on a beta counter at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor alone was 4.330 μM. The IC₅₀ of the CDK4 inhibitor together with the alanosine was 0.9955 μM. The IC₅₀ of the CDK4 inhibitor together with the alanosine and with adenine was 5.564 μM. The IC₅₀ of the CDK4 inhibitor together with the adenine was 7.356 μM. See FIG. 2.

### Example 4

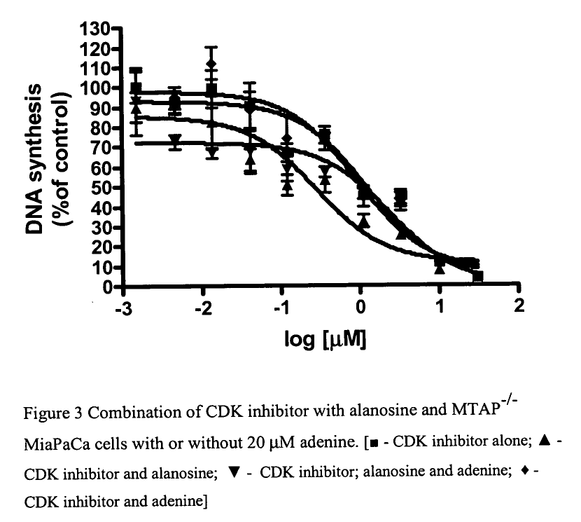

Combination of de novo purine biosynthesis inhibitor and CDK4 inhibitor in MTAP^{-/-} cells with adenine. MTAP^{-/-} MiaPaCa-2 cells from Example 1 were seeded at 2×10⁴ cells/ml in 100 μl per well in Costar T plates (Amersham Biosciences). Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) was added to triplicate wells with or without 1.7 μM (IC₂₀) of alanosine. Some cells were also treated with 20 μM adenine (Sigma) as rescue substrate. ¹⁴C-Thymidine (0.1 μCi, Amersham) was added to each well for a final total volume of 200 μL. Plates were read for incorporation of the radioactive thymidine as a measure of cell viability on a beta counter at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor alone was 1.276 μM. The IC₅₀ of the CDK4 inhibitor together with the alanosine was 0.2866 μM. The IC₅₀ of the CDK4 inhibitor together with the alanosine and with adenine was 2.458 μM. The IC₅₀ of the CDK4 inhibitor together with the adenine was 0.9495 μM. See FIG. 3.

### Example 5

Combination of de novo purine biosynthesis inhibitor and CDK4 inhibitor in MTAP^{+/+} cells with MTA. MTAP^{+/+} MiaPaCa-2 cells from Example 1 were seeded at 2×10⁴ cells/ml in 100 μl per well in Costar T plates (Amersham Biosciences).

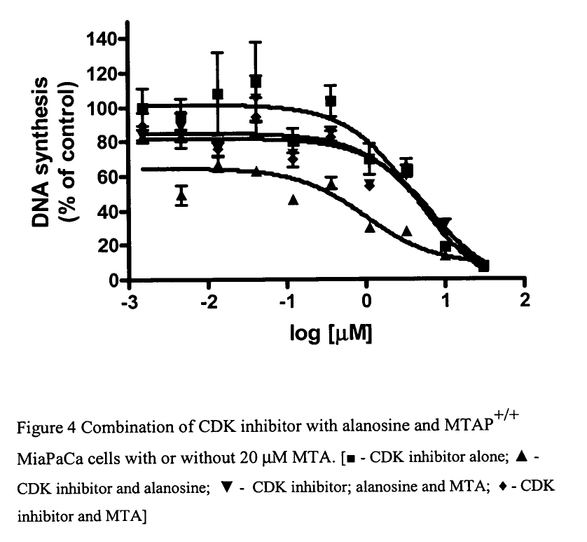

Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) was added to triplicate wells with or without 1.7 μM (IC₂₀) of alanosine. Some cells were also treated with 20 μM MTA (Sigma) as rescue substrate. ¹⁴C-Thymidine (0.1 μCi, Amersham) was added to each well for a final total volume of 200 μL. Plates were read for incorporation of the radioactive thymidine as a measure of cell viability on a beta counter at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor alone was 4.330 μM. The IC₅₀ of the CDK4 inhibitor together with the alanosine was 0.9955 μM. The IC₅₀ of the CDK4 inhibitor together with the alanosine and with MTA was 6.104 μM. The IC₅₀ of the CDK4 inhibitor together with the MTA was 8.253 μM. See FIG. 4.

### Example 6

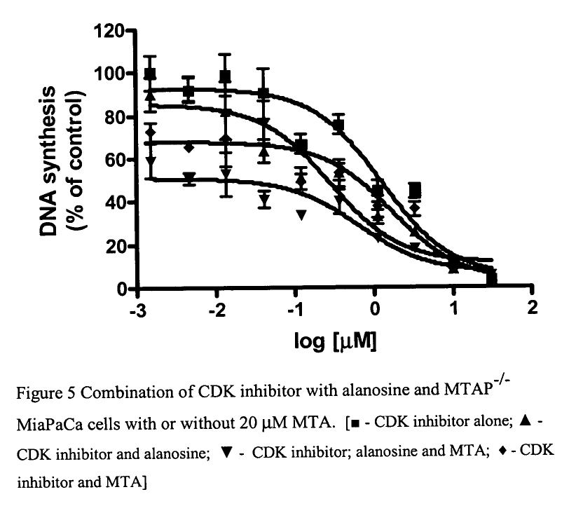

Combination of de novo purine biosynthesis inhibitor and CDK4 inhibitor in MTAP^{-/-} cells with MTA. MTAP^{-/-} MiaPaCa-2 cells from Example 1 were seeded at 2×10⁴ cells/ml in 100 μl per well in Costar T plates (Amersham Biosciences). Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) was added to triplicate wells with or without 1.7 μM (IC₂₀) of alanosine. Some cells were also treated with 20 μM MTA (Sigma) as rescue substrate. ¹⁴C-Thymidine (0.1 μCi, Amersham) was added to each well for a final total volume of 200 μL. Plates were read for incorporation of the radioactive thymidine as a measure of cell viability on a beta counter at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor alone was 1.276 μM. The IC₅₀ of the CDK4 inhibitor together with the alanosine was 0.2866 μM. The IC₅₀ of the CDK4 inhibitor together with the alanosine and with MTA was 0.6432 μM. The IC₅₀ of the CDK4 inhibitor together with the MTA was 1.707 μM. See FIG. 5.

### Example 7

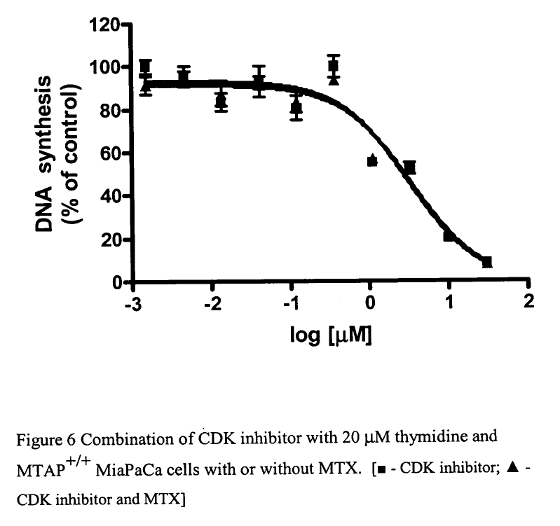

Combination of MTX and CDK4 inhibitor in MTAP^{+/+} cells. MTAP^{+/+} MiaPaCa-2 cells from Example 1 were seeded at 2×10⁴ cells/ml in 100 μl per well in Costar T plates (Amersham Biosciences). Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) and thymidine (20 μM, Sigma) was added to triplicate wells with or without 20 nM (IC₂₀) of MTX. ¹⁴C-Thymidine (0.1 μCi, Amersham) was added to each well for a final total volume of 200 μL. Plates were read for incorporation of the radioactive thymidine as a measure of cell viability on a beta counter at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor alone was 3.2 μM. The IC₅₀ of the CDK4 inhibitor together with MTX was 3.5 μM. See FIG. 6.

### Example 8

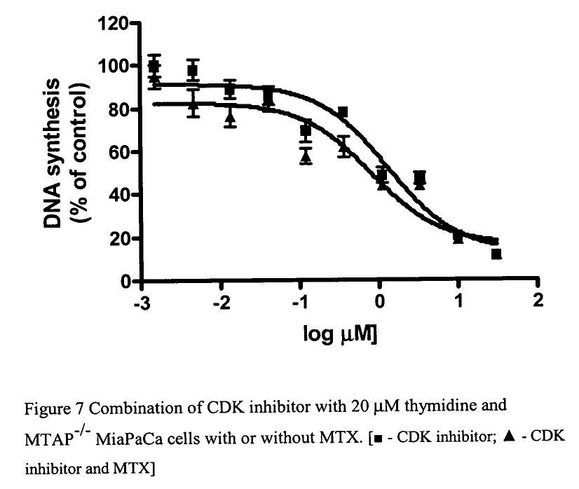

Combination of MTX and CDK4 inhibitor in MTAP^{-/-} cells. MTAP^{-/-} MiaPaCa-2 cells from Example 1 were seeded at 2×10⁴ cells/ml in 100 μl per well in Costar T plates (Amersham Biosciences). Twenty-four hours later a dose response of a CDK4 inhibitor PD-0332991 (1 nM; 3 nM, 10 nM, 30 nM, 100 nM, 300 nM, 1 μM, 3 μM, 10 μM and 30 μM concentrations) and thymidine (20 μM, Sigma) was added to triplicate wells with or without 20 nM (IC₂₀) of MTX. ¹⁴C-Thymidine (0.1 μCi, Amersham) was added to each well for a final total volume of 200 μL. Plates were read for incorporation of the radioactive thymidine as a measure of cell viability on a beta counter at time 0, 24, 48, 72 and 96 hours. Data was calculated as the mean and standard deviation of triplicate samples divided by the mean and standard deviation of untreated control samples to yield percent of control. The IC₅₀ of the CDK4 inhibitor alone was 1.3 μM. The IC₅₀ of the CDK4 inhibitor together with MTX was 0.8 μM. See FIG. 7.

Alanosine shifted the IC₅₀ of the CDK4 inhibitor in both MTAP^{+/+} and MTAP^{-/-} cells. This shift was rescued with adenine in both cell lines and with MTA in the MTAP^{+/+} cells. The IC₅₀'s of the CDK4 inhibitor between the two cell lines was different from the first experiment and the MTAP^{+/+} cells seemed to be a bit more resistant. MTX did not significantly shift the IC₅₀ of the CDK4 inhibitor in the MTAP^{+/+} cells.

It is believed that dosing sequence, where the de novo inhibitor is administered prior to the CDK inhibitor will provide more beneficial effect.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

No unacceptable toxological effects are expected when combinations of the present invention are administered in accordance with the present invention.

All mentioned references, patents, applications and publications, are hereby incorporated by reference in their entirety, as if here written.

What is claimed:

1. A pharmaceutical composition comprising a de novo purine biosynthesis inhibitor and at least one CDK inhibitor.

2. The pharmaceutical composition of claim 1 containing a CDK4 inhibitor.

3. The pharmaceutical composition of claim 1 containing a CDK6 inhibitor.

4. The pharmaceutical composition of claim 1 wherein the CDK inhibitor is selected from P-276-00, GW-491619, NU-6027, AG-12275, AG-12286, PD-0166285, PD-0332991 and Alvocidib.

5. The pharmaceutical composition of claim 1 wherein the de novo purine biosynthesis inhibitor inhibits AdSL.

6. The pharmaceutical composition of claim 1 wherein the de novo purine biosynthesis inhibitor inhibits AdSS.

7. The pharmaceutical composition of claim 1 wherein the de novo purine biosynthesis inhibitor is selected from alanosine and SDX-102.

8. The pharmaceutical composition of claim 1 wherein the de novo purine biosynthesis inhibitor is methotrexate.

9. A method of treating cancer with a combination comprising at least one de novo purine biosynthesis inhibitor and at least one CDK inhibitor.

10. The method of claim 9 comprising a rescue substrate.

11. The method of claim 10 wherein the rescue substrate is adenine, MTA or an MTA derivative.

12. The method of claim 10 wherein de novo purine biosynthesis inhibitor is administered after the CDK inhibitor.

13. A kit comprising, in one or more containers, separately or in admixture one or more de novo purine biosynthesis inhibitor and at least one CDK inhibitor.

14. A method of detecting tumors having p16 and MTAP co-inactivation, wherein the inactivation identifies a tumor that is likely to respond to a composition comprising at least one de novo purine biosynthesis inhibitor and at least one CDK inhibitor.

15. A method of treating a subject determined to have tumors with inactivation of both p16 and MTAP.

16. The method of claim 15, wherein the tumors have inactivation of both p16-related polynucleotide or polypeptide and MTAP polynucleotide or polypeptide.

17. A method for prognostic or diagnostic assessment of a neoplastic disorder in a subject, comprising: a) preparing a sample of nucleic acids from a specimen obtained from the subject; b) contacting the sample with a panel of nucleic acid segments consisting of at least 2 members from the group consisting of p16, CDK4, CDK6, and MTAP to detect the levels of the panel segments; c) evaluating the sample against a reference standard to determine the magnitude of change in the amounts of the at least 2 members present in the sample; and d) correlating the magnitude of change with the presence or resolution of the disorder.
